(19) United States
(12) Patent Application Publication
(10) Pub. No.: US 2012/0271345 A1
(43) Pub. Date: Oct. 25, 2012

(54) APPARATUS AND METHOD FOR TREATING MYOFASCIAL TRIGGER POINTS

(75) Inventor: Jonathan K. Torres, Yarmouth, ME (US)

(73) Assignee: CLIPPER PROFESSIONAL MEDICAL SERVICES LLC, Yarmouth, ME (US)

(21) Appl. No.: 13/241,969

(22) Filed: Sep. 23, 2011

Related U.S. Application Data
(60) Provisional application No. 61/478,178, filed on Apr. 22, 2011.

(51) Int. Cl. A61B 17/00

(52) U.S. Cl. 606/204

(57) ABSTRACT

A device and method for the application of pressure to a location where muscle pain exists, including at a trigger point. The device and method provide gradual, uniform and systematic progression of pressure and release. The device and method enable the application of pressure in a manner that allows a patient to tolerate the pressure without tensing the relevant muscles and/or causing the locus of pain to become hyperirritable and painful. The device and method also enable the gradual selectable increase in that applied pressure while still preventing the tense response from the patient so that the optimal or desired amount of focused pressure is tolerated. The device enables progressive deep tissue massage in systematic and progressive steps that enhances the treatment of myofascial trigger points in skeletal muscles and other forms of muscle pain. The device includes a plurality of terminal configurations that differ in hardness and/or dimensions.

### CROSS REFERENCE TO RELATED APPLICATION

This application claims the priority benefit of U.S. provisional application Ser. No. 61/478,178 filed Apr. 22, 2011, entitled APPARATUS AND METHOD FOR TREATING MYOFASCIAL TRIGGER POINTS, of the same named inventor. The entire content of the priority application is incorporated herein by reference.

### FIELD OF THE INVENTION

The present invention relates generally to an apparatus and method for treating myofascial trigger points. More specifically, the apparatus is configured to enable a patient to self-treat myofascial trigger points through the progressive application of different pressures to the trigger points through massage and/or trigger point pressure and release of that pressure. The related method provides particular steps associated with the use of the apparatus.

### BACKGROUND OF THE INVENTION

The fibrous tissue that encloses and separates muscle layers is identified as myofascial tissue. A myofascial trigger point is a hyper-irritated area of that tissue, characterized as a taut band of muscle that is very tender with compression and often radiates pain to other areas of the body. Trigger points can be latent, causing stiffness, posture distortion, and motion restriction, or active, causing pain and immobility. This issue is most common among middle-aged adults and can result from many different causes including, but not limited to, muscle strains, stress, overuse, trauma, and poor nutrition. Small tears in the soft muscle tissue can occur when a muscle fiber, tendon, or ligament is overstretched or inflamed. A muscle unit contracts in attempt to heal and protect itself, thus restricting the movement of the muscle at least in that location. This is the process of development of the trigger points, which can form on any of the 400 muscles that make up the skeletal muscle and commonly involve the upper back/trapezius musculature. Individuals with myofascial pain syndrome and related trigger points experience pain in muscle areas, muscle knots and decreased range of motion of the muscle. They also experience referred pain to the extremities, ringing in the ear, temperomandibular joint pain and headaches.

Trigger points result in restricted blood flow (ischemia) through the muscle in that vicinity of the trigger point. That blood flow restriction produces significant pain, restricted motion and disability in a person. Each person who experiences the pain, restriction, and discomfort of trigger points has a unique situation because trigger points are unique in size, tenderness, location and development.

Currently, there are many ways for trigger points to be treated. These ways include physical therapy, massage therapy, trigger point injections, osteopathic manipulation, acupuncture and pharmacological treatment. Unfortunately, many patients do not respond well to current treatment mechanisms. As a result, long-term symptoms have not been sufficiently addressed for a sufficient number of people.

Massage treatment appears to provide a suitable avenue for dealing with trigger point difficulties. Massage treatment for trigger points involves trigger point pressure release, which has also been referred to as ischemic compression. Trigger point pressure release is the application of steady pressure to the trigger point with sufficient force and duration to loosen knotted fibers and allows fresh blood to travel more freely into the affected area, or at least not to diminish further the blood flow, and then the release of that pressure. The pressure is applied until a barrier of pain is reached and then more pressure is applied until the next pain barrier is reached. Ideally, only enough pressure is applied until the pain threshold of the patient is reached and no more. The patient's pain tolerance gradually increases, or the contraction knot of the trigger point releases, to an extent, and then more pressure can be applied at the next stage of the process. Unfortunately, this process of increased pressure application generally occurs all within the same session and is therefore not well associated with the particular patient's condition and needs. The pressure/release may be delivered by a health care provider or by the patient through self massage. However, current massage treatment can be less than optimal due to a lack of, or excessive, intensity. Further, some trigger points are located in places that are too difficult to thoroughly massage. Also, suitably reliable pressure may not always be applied to the right location for a sufficient period of time. Massage treatment may also be too expensive for some patients to rely upon for a sufficient length of time to address adequately the myofascial pain suffered by the patient.

In those situations where the trigger point pressure/release is properly employed, it may nevertheless fail to provide desired relief if the trigger point is too irritable, the person applying the pressure presses too hard given the sensitivity, and the patient involuntarily tenses the relevant muscle in response to the pain. That tensing seals the trigger point from the pressure needed to release contraction knots, rendering the treatment ineffective. Therefore, the hypersensitivity of trigger points makes massage therapy that would otherwise be effective inadequate in treating trigger points in many instances. While the use of trigger point pressure/release is desirable, the current massage therapy, self massage and/or the use of assistive massage devices, such as, for example, but not limited thereto, the Thera Cane™ available from the Thera Cane Company of Denver, Colo., and the Knobble™ II hand held massager and the Backnobber II product, both available from The Pressure Positive Co., of Gilbertsville, Pa., options available to patients are not satisfactory.

The Thera Cane™ product is a self massager that comprises a curved bar with extensions spaced at several locations along the length of the bar. The extensions include rounded terminals that the patient applies to one or more trigger points. The patient applies pressure to that location using leverage applied to the curved bar. The Backnobber™ II product is a double-curved bar that is designed to allow a user to apply a rounded terminal to the back by hooking a first one of the curves over the shoulder and pressing on the second one of the curves to apply pressure to a location of interest. The Thera Cane™, Knobble™ II and Backnobber™ II can provide some beneficial effect; however, the fixed rounded terminals are uniform and of a single dimension. They are to be applied to all trigger points, regardless of the particular attributes of those trigger points, including location, size and depth. As a result, the patient is forced to apply the same type of pressure, as a function of the terminal diameter and fixed hardness, to all trigger points. This often leads to the patient's reluctance to use it effectively, as the level of pressure may be too much or not enough and can generate muscle tensing at a trigger point when the device, even when used as instructed, causes excessive pain.

What is needed is a better device and related method for the delivery of trigger point pressure. The device and method should provide for the tolerable application of trigger point pressure and then release. The device and method should provide the pressure in this way to allow the patient to tolerate the pressure without tensing the relevant muscles. Further, the device and method should allow for the gradual increase in that pressure while still preventing the tense response from the patient so that the desired release of contraction knots in the trigger point is achieved, resulting in a release of muscle tension in one or more taut muscle bands. The patient is thereby able to tolerate progressive trigger point compression and therefore will likely be more compliant with treatment.

### SUMMARY OF THE INVENTION

The present invention is a device and related method for the delivery, including the optional self-delivery, of trigger point pressure. The device and method enable selective application of trigger point pressure, which pressure may be gradual, uniform and systematic but is not limited thereto. The device and method enable the application of trigger point pressure in a manner that allows a patient to tolerate the applied pressure without tensing the relevant muscles and without causing the trigger point to become hyperirritable. The device and method also enable the gradual selectable increase in that applied pressure while still preventing the tense response from the patient so that the optimal pressure is achieved. The device enables progressive deep tissue massage in systematic and progressive steps that enhances the treatment of myofascial trigger points in skeletal muscles and improves patient compliance. It is also designed to remove from the patient the obligation to know the amount of pressure to be applied when self massage is performed. The systematic and/or progressive approach provided by the present invention enables a finer incremental increase in the application of pressure, which granularity is not provided by any of the presently available trigger point pressure application devices or processes. This is as applicable for patients performing a self massage procedure as it is for any healthcare provider who treats patients with myofascial trigger points.

One embodiment of the device of the present invention is a massager formed of a primary body with one or more handles and one or more extensions. The massager may be used by the patient in a self massaging fashion, or by a care giver who treats the patient. Each of the one or more extensions includes a pressure application terminal. One or more of the terminals may be of a changeable dimension. Alternatively, in an embodiment of the invention wherein the primary body includes a plurality of terminals, the terminals may or may not be changeable. In an embodiment of the invention wherein not all of the terminals are changeable, the terminals are of different dimensions and/or hardnesses. Contrary to the Thera Cane™, Knobble™ II and Backnobber™ II products, the massager of the present invention includes an arrangement to allow a person to apply pressure using different surface area dimensions. In particular, the device of the present invention enables the person to massage the patient in a suitable way to apply trigger point pressure appropriate for that patient's condition, including the status of the trigger point; i.e., size and sensitivity. In an embodiment of the invention, the terminals may be different in both hardness and dimensions. They may also be different by hardness only or dimensions, such as circumference, only. In another embodiment of the invention, the terminals may be of changeable shape. For example, one may began treatment with a rounded surface and progress to a pointed surface, or such other shape changing progression suitable for changing the applied pressure in a selectable manner.

The present invention provides a person with a customized way to address a patient's particular condition through a progressive, gradual or other suitable form of application of pressure at the trigger point. In general, it is expected that the device terminal that is softest and/or with the largest contact surface area is directed to the trigger point. That softest/largest terminal limits the force applied at the trigger point so that even if substantial pressure is applied, the force of that compression is either partially absorbed at the terminal or is spread across a relatively wide area. The patient therefore has confidence that desired trigger point compression will occur without causing the pain that may result in muscle tensing. As the patient's comfort level grows, the device terminal used to apply the pressure is either relatively harder or of relatively smaller dimension than the first terminal. The resultant pressure at the site is greater than applied in the first stage described when the patient pushes with the same force as used in the first stage. This process can be continued for a plurality of iterations until the patient is satisfied that the myofascial pain has been sufficiently reduced at the trigger point. It is to be understood that any combination of terminal hardness and dimensional variations form an aspect of the invention and may be provided in multiple devices, each appropriate to address the unique condition of each patient. It is to be noted that a healthcare provider may first assess the patient's sensitivity, likely with a dolorimeter and/or measuring the size of the trigger point, and then make a recommendation regarding the particular course of treatment required by the patient using a selectable progression that may or may not begin with the softest/largest device terminal.

The device and related method of the present invention allows a patient to undergo the process of healing at a pace that is both healthy and individually beneficial in a progressive fashion. Pressure is applied more firmly and with more focus in a selectably incremental manner as the trigger point becomes smaller and less hyperirritable. This increases the likelihood for successful resolution of the myofascial pain/trigger point condition. The invention improves on existing devices and massage procedures because it decreases the likelihood of making the trigger point more painful, thereby improving patient tolerance and compliance. The device and method allow the patient to progress on whatever schedule is suitable for him or her. The progression may be carried out over any time period, including over the course of a single treatment session. This customizable progression at refinable increments allows for successful treatment and improved outcomes for the myofascial pain condition. More patients are likely to recover and experience improvement in their quality of life as the device and related method of use prevent, or at least substantially reduce the likelihood, of muscle tensing, a pain response and aggravation of the trigger point.

The present invention is a device for treating muscle pain that may include one or more trigger points, the device comprising a base device including a terminal and means for causing movement of the base device and one or more end caps removably placeable on the terminal, wherein the one or more end caps are of one or more different physical characteristics than the terminal and each other. The one or more different physical characteristics may be selected from hardness, dimension and shape. The invention is also a kit for treating muscle pain that may include one or more trigger points, the kit comprising a first device including a terminal and means for causing movement of a first base device and a second device including a terminal and means for causing movement of a second base device, wherein the terminal of the first base device has one or more physical characteristics different from one or more physical characteristics of the second base device, and wherein the one or more different physical characteristics are selected from hardness, dimension and shape. The invention is also a method for treating muscle pain that may include one or more trigger points, the method comprising the steps of applying pressure to an area where the muscle pain exists using a device having a terminal of a first one or more physical characteristics, releasing the pressure applied with the device having the terminal of the first one or more physical characteristics, changing the terminal of the device so that it is of a second one or more physical characteristics, wherein the second one or more physical characteristics are different from the first one or more physical characteristics and applying pressure to the area where the muscle pain exists using the device with the second hardness and/or second dimension, wherein the first one or more different physical characteristics and the second one or more different physical characteristics may be selected from hardness, dimension and shape. The invention is also a method for treating muscle pain that may include one or more trigger points, the method comprising the steps of applying trigger point pressure release to a trigger point using a first device having a terminal of a first one or more different physical characteristics and applying trigger point pressure release to the trigger point using a second device with a second one or more different physical characteristics, wherein the first one or more different physical characteristics the second one or more different physical characteristics are not equal, and wherein the first one or more different physical characteristics and the second one or more different physical characteristics may be selected from hardness, dimension and shape. These and other advantages of the present invention will become apparent upon review of the following detailed description of preferred embodiments and the accompanying drawings.

### BRIEF DESCRIPTION OF THE DRAWINGS

FIG. 1 is a top view of a first embodiment of the device of the present invention.

FIG. 2 is a top view of the plurality of end caps that may be removably applied to the base terminal of the device shown in FIG. 1.

FIG. 3 is a top view of a second embodiment of the device of the present invention.

FIG. 4 is a top view of a third embodiment of the present invention, which may be used with the end caps shown in FIG. 2.

FIG. 5 is a top view of a fourth embodiment of the present invention, which may be used with the end caps shown in FIG. 2, showing one of the end caps adjacent thereto.

FIG. 6 is a top view of the fourth embodiment of the present invention, showing the end cap of FIG. 5 applied thereon.

FIG. 7 is a flow chart illustrating the steps of the method of the present invention.

### DETAILED DESCRIPTION OF THE PREFERRED EMBODIMENTS

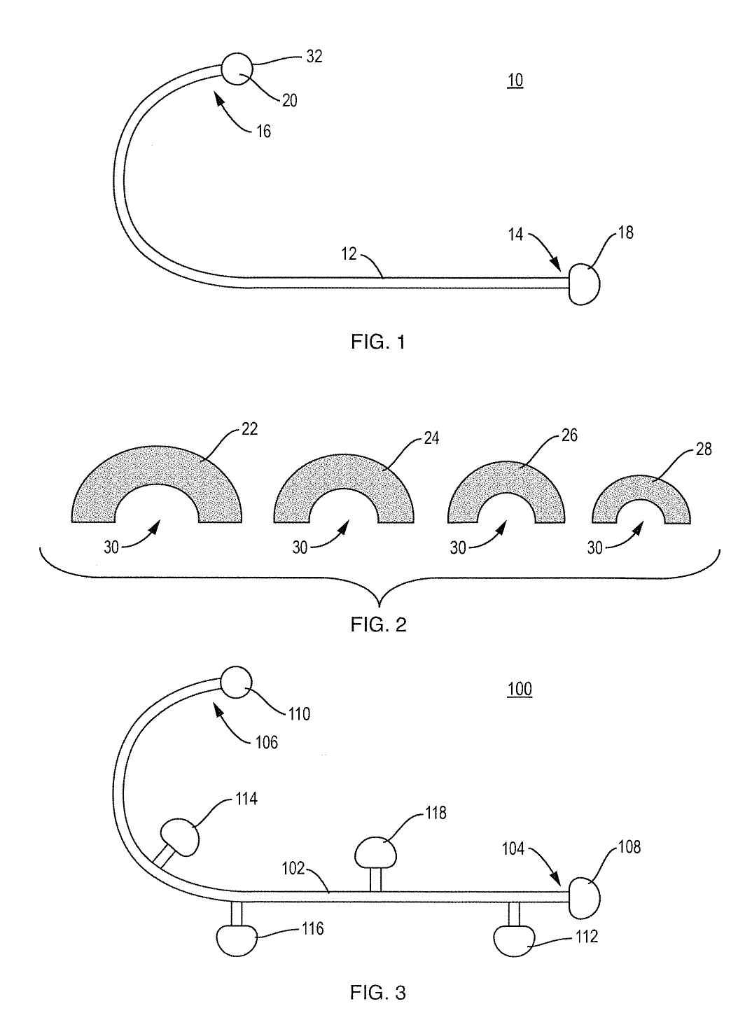

The present invention is a massage device and method for the self treatment of myofascial pain and one or more associated trigger points. While this description of the preferred embodiment of the invention is directed to the treatment of trigger points, it is to be understood that is equally applicable to the treatment of any sort of muscle pain, muscle tightness or any other condition for which the application and release of pressure in a gradual, progressive and/or selectable way is considered to be beneficial. A first embodiment of a massage device 10 is shown in FIG. 1. The device 10 includes a primary body 12 with a first end 14 and a second end 16. The first end 14 includes a knob 18 that a patient may hold onto when employing the device 10. The second end 16 includes a terminal base 20. The terminal base 20 is shown as rounded but is not limited to that shape. The device 10 may be formed as one or more pieces and may be fabricated of a metal material or a non-metallic material including, but not limited to, a viscoelastic material. The primary body 12 of the device 10 is shown in a curved arrangement to facilitate a user's application of the terminal base 20 to the user's back by pressing on the knob 18. In that respect, the device 10 shown is similarly shaped to the Thera Cane™ product previously described. Alternatively, the primary body 12 may be straight or any other configuration considered suitable for a patient to use as a self massager.

The device 10 of the present invention further includes one or more end caps, which are shown in FIG. 2. In an embodiment of the invention, there are four end caps, first end cap 22, second end cap 24, third end cap 26 and fourth end cap 28. Each of the end caps includes an opening 30, which is sized to permit removable attachment of the end cap to the terminal base 20 of the device 10. One or more of the end caps may be flexible to permit a snug fit and close interface contact with surface 32 of the terminal base 20. Alternatively, one or more of the end caps may be relatively rigid and fabricated to establish a reasonable removable fit with the terminal base 20. One or more of the end caps may be fabricated of foam. Additionally, one or more of the end caps may be fabricated of a solid material including rubber and plastic. Surface texture may be selectable, including smooth and rough as basic options. The device 10, the terminal base 20 and/or the end caps may be color coded for ease of use. It is to be noted that more or fewer end caps may be used, provided the opportunity to make a progression from softer and/or larger to harder and/or smaller is provided. In other words, while the invention is described herein with respect to four progression options in addition to the fixed terminal base, it is to be understood that more or less than four end caps may be employed.

In the preferred embodiment of the invention, each of the end caps is a different hardness. Further, the end caps are configured with a progression of hardness. For example, the first end cap 22 may be the softest and fourth end cap 28 may be the hardest, with the second end cap 24 harder than the first end cap 22 and the third end cap 26 being harder than the second end cap 24. The increasing hardness may be linear or non-linear. In another embodiment of the invention, the end caps may all be of substantially the same hardness but they may be sized differently, wherein the first end cap 22 may have the greatest surface area and the fourth end cap 28 may have the smallest surface area, with the second end cap 24 having a surface area smaller than that of the first end cap 22 and the third end cap 26 having a surface area smaller than that of the second end cap 24. The changes in surface area dimensions may be linear or non-linear from one end cap to the next. The end caps may be color-coded and/or numbered to aid in resolving which end cap to use when during the course of a treatment. For example, first end cap 22 may be yellow, second end cap 24 may be red, third end cap 26 may be green and fourth end cap 28 may be orange. Any color coding that may be employed is not limited to these particular colors or this particular order of colors. This color coding may help in the understanding of which end cap to use at what point of the therapy procedure. It is to be noted that the color coding is not limited to the end caps and may also be used in association with the device 10 and/or the terminal base 20 as well. The patient preferably begins a therapy procedure with the softest or largest end cap to begin, the first end cap 22, and may finish the therapy procedure with the hardest or smallest end cap, end cap 28.

The end caps, when applied to the terminal base 20, regulate the trigger point pressure that is applied by the patient to the trigger point. Whereas the terminal base 20 alone would transfer a substantial portion of the force applied by the patient's push on the knob 18, the end cap placed intermediate between the terminal base 20 and the patient's body enables the modification of that force. When the softest/largest first end cap 22 is used, the greatest portion of the force is absorbed (softest end cap) or most widely distributed (largest end cap). Progressing from application of the first end cap 22 to the terminal base 20 to the fourth end cap 28 to the terminal base 20 results in an increase in the trigger point pressure to the trigger point where the device 10 is applied. It is to be noted that more or fewer end caps may be provided. It is also to be noted that the last step of the process may involve removal of the fourth end cap 28 and use of the terminal base 20 directly at the area to be treated.

The progression of the end caps by hardness may range, for example, from a Shore A durometer hardness of about 35 for the first end cap 22 to a Shore A hardness of about 40 for the second end cap 24, to a Shore A hardness of about 45 for the third end cap 26 and to a Shore A hardness of about 50 for the fourth end cap 28. The present invention is not limited to that progression. When the end cap progression is provided by changes in dimensions and the terminal base 20 has a circumference of about one inch, the end cap dimensions may range, for example, from a circumference of about 2.375 inches for the first end cap 22, to a circumference of about 2.045 inches for the second end cap 24, to a circumference of about 1.705 inches for the third end cap 26 and to a circumference of about 1.375 inches for the fourth end cap 28. The present invention is not limited to that progression. It is to be noted that the end caps may also progress from softest and largest to hardest and smallest. In an embodiment of the invention, the dimensions may be different in a manner that also produces differences in hardness. For example, the thicknesses of the end cap walls may progress from about 0.628-inch thick for end cap 22, to about 0.464-inch thick for end cap 24, to about 0.293-inch thick for end cap 26 and to about 0.129-inch thick for end cap 28. Of course, these dimensions, hardnesses and wall thicknesses may be adjusted as desired without deviating from the progressive aspect of the invention.

In an alternative embodiment of the device 10, there may be provided to a patient a plurality of such devices wherein each of the devices in a set of such devices includes a terminal base 20 having a different hardness and/or dimension. That is, each alternative of device 10 of the present invention may have a terminal base 20 configured in the manner of the end caps 22/24/26/28 so that the patient may switch from one such device of the set to the next in the progression of the treatment of the trigger point.

A second embodiment of the invention is shown in FIG. 3. A second massage device 100 includes a primary body 102 with a first end 104 and a second end 106. The first end 104 includes a knob 108 that a patient may hold onto when employing the device 100. The second end 106 includes a terminal base 110. The primary body 102 also includes a plurality of pressure bases, a first pressure base 112, a second pressure base 114, a third pressure base 116 and a fourth pressure base 118. Each of the terminal base 110 and the pressure bases is shown as rounded but they are not limited to that shape. The device 100 may be formed as one or more pieces and may be fabricated of a metal material or a non-metallic material including, but not limited to, a viscoelastic material. The primary body 102 of the device 100 is shown in a curved arrangement to facilitate a user's application of the terminal base 110 and one or more of the pressure bases to the user's back by pressing on the knob 108. In that respect, the device 100 shown is similarly shaped to the Thera Cane™ product previously described. Alternatively, the primary body 102 may be straight or any other configuration considered suitable for a patient to use as a self massager.

In an embodiment of the device 100, each of the pressure bases may be of substantially the same hardness but they are sized differently, wherein the first pressure base 112 may have the greatest surface area and the fourth pressure base 118 may have the smallest surface area, with the second pressure base 114 having a surface area smaller than that of the first pressure base 112 and the third pressure base 116 having a surface area smaller than that of the second pressure base 114. The changes in surface area dimensions may be linear or non-linear from one pressure base to the next. The patient preferably begins a therapy procedure with the largest pressure base, the first pressure base 112, and may finish the therapy procedure with the smallest pressure base, the fourth pressure base 118. Alternatively, the hardnesses of the pressure bases, as well as the terminal base 110, may vary from one to another. Color coding may be employed in this embodiment with respect to any one or more of the device 100, the terminal base 110 and the pressure bases.

The progression of the pressure bases may range, for example, from a diameter of about 2.375 inches for the first pressure base 112, to a diameter of about 2.045 inches for the second pressure base 114, to a diameter of about 1.705 inches for the third pressure base 116 and to a diameter of about 1.375 inches for the fourth pressure base 118. The present invention is not limited to that progression. In that progression, the terminal base 110 may also be used as a pressure application element. It is to be noted that the end caps of FIG. 2 may also optionally be used on one or more of the pressure bases and/or the terminal base 110. It is to be noted that more or pressure bases may be used, provided the opportunity to make a progression from softer and/or larger to harder and/or smaller is provided. In other words, while the invention is described herein with respect to four progression options in addition to the fixed terminal base, it is to be understood that more or less than pressure bases may be employed.

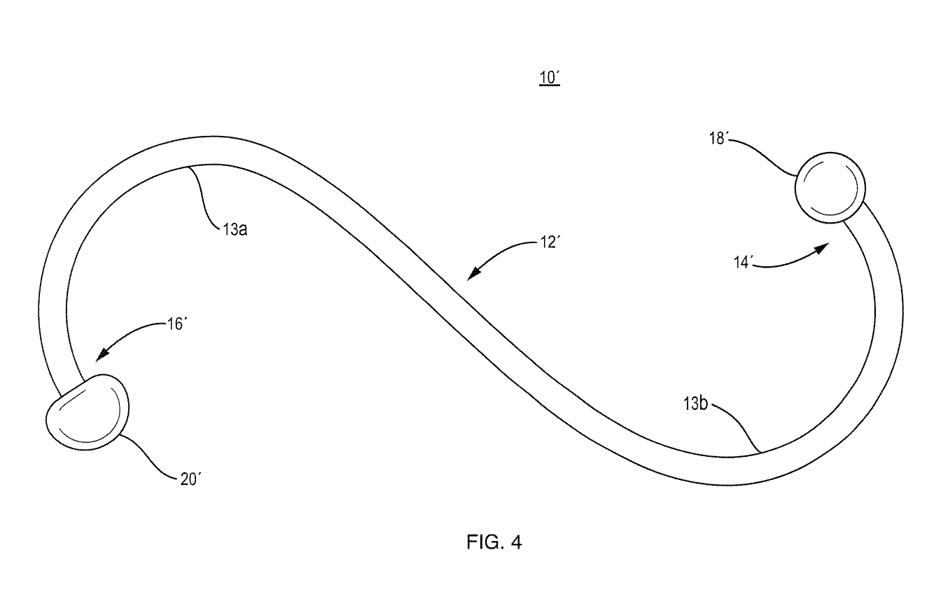

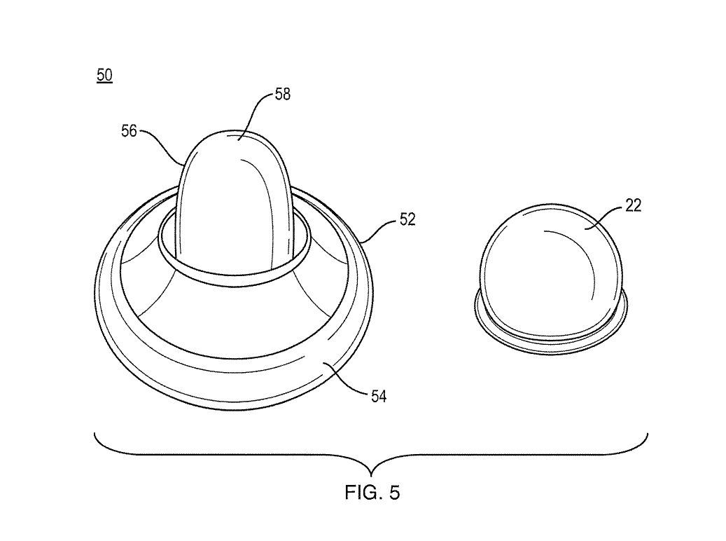

A third embodiment of a massage device 10′ is shown in FIG. 4. The device 10 includes a primary body 12′ with a first end 14′ and a second end 16′. The first end 14′ includes a knob 18′ that a patient may hold onto when employing the device 10′. The second end 16′ includes a terminal base 20′. The terminal base 20′ is shown as rounded but is not limited to that shape. The device 10′ may be formed as one or more pieces and may be fabricated of a metal material or a non-metallic material including, but not limited to, a viscoelastic material. The primary body 12′ of the device 10′ is shown in a double curved arrangement to facilitate a user's application of the terminal base 20′ to the user's back by hooking first curve 13a over the shoulder and pressing on second curve 13b and/or pressing on the knob 18′. In that respect, the device 10 shown is similarly shaped to the Backnobber™ II product described above modified to include the end caps described herein removably applied to the terminal base 20′.

The device 10′ of FIG. 4 is used in combination with the end caps shown in FIG. 2 and previously described herein. Specifically, each of the end caps is a different hardness and/or a different dimension. The end caps, when applied to the terminal base 20′, regulate the force that is applied by the patient to the trigger point. Whereas the terminal base 20′ alone would transfer a substantial portion of the force applied by the user's push on the knob 18′, the end cap placed intermediate between the terminal base 20′ and the patient's body enables the modification of that force. When the softest/largest first end cap 22 is used, the greatest portion of the force is absorbed (softest end cap) or most widely distributed (largest end cap). Progressing from application of the first end cap 22 to the terminal base 20′ to the fourth end cap 28 to the terminal base 20′ results in an increase in the trigger point pressure applied at the trigger point. It is to be noted that more or fewer end caps may be provided. It is also to be noted that the last step of the process may involve removal of the fourth end cap 28 and use of the terminal base 20′ directly at the area to be treated. Color coding may be employed in this embodiment with respect to one or more of the device 10′, the terminal base 20′ and the end caps.

In an alternative embodiment of the device 10′ of FIG. 4, there may be provided to a patient a plurality of such devices wherein each of the devices in a set of such devices includes a terminal base 20′ having a different hardness and/or dimension. That is, each alternative of device 10′ of the present invention may have a terminal base 20′ configured in the manner of the terminal ends 110/112/114/116/118 of the device 100 of FIG. 3 so that the patient may switch from one such device of the set to the next in the progression of the treatment of the trigger point. Color coding may be employed in this alternative embodiment with respect to one or more of the device 10′, the terminal base 20′ and the terminal ends.

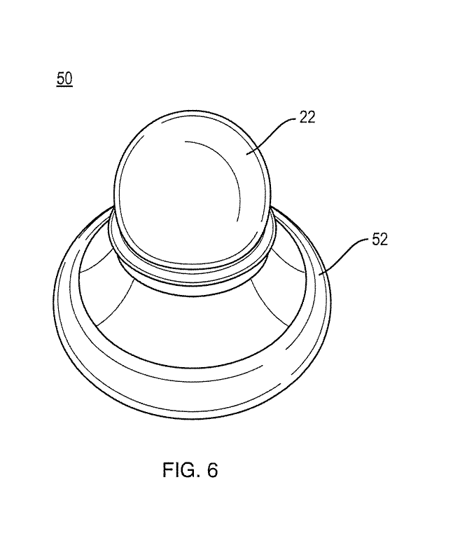

A fourth embodiment of a massage device 50 of the present invention is shown in FIGS. 5 and 6. The device 50 is a hand-held device including a primary body 52 having a base 54 in a rounded cylindrical shape, and a post 56 of rounded shape affixed to the base 54 and extending from a center portion of the base 54. The base 54 is configured to enable a person to grasp it in one hand, apply pressure to a trigger point at post end 58 and rotate the device 50 as instructed by a healthcare giver or as desired. The device 50 may be formed as one or more pieces and may be fabricated of a metal material or a non-metallic material including, but not limited to, a viscoelastic material. The device 50 shown is similarly shaped to the Knobble™ II hand held massager identified above, and modified to include the end caps described herein removably applied to the post end 58.

The device 50 of FIG. 5 is used in combination with the end caps shown in FIG. 2 and previously described herein. An example of one of the end caps is first end cap 22 shown in FIG. 5 spaced away from the device 50 and removably applied to the post end 58 in FIG. 6. The end caps, when applied to the post end 58, regulate the force that is applied by the patient to the trigger point. Whereas the post end 58 alone would transfer a substantial portion of the force applied by the patient's push on the base 54, the end cap placed intermediate between the post end 58 and the patient's body enables the modification of that force. When the softest/largest first end cap 22 is used, the greatest portion of the force is absorbed (softest end cap) or most widely distributed (largest end cap). Progressing from application of the first end cap 22 to the post end 58 to the fourth end cap 28 to the post end 58 results in an increase in the trigger point pressure where applied. It is to be noted that more or fewer end caps may be provided. It is also to be noted that the last step of the process may involve removal of the fourth end cap 28 and use of the post end 58 directly at the area to be treated. Color coding may be employed with this embodiment with respect to one or more of the device 50, the post end 58 and the end caps.

In an alternative embodiment of the device 50 of FIGS. 5 and 6, there may be provided to a patient a plurality of such devices wherein each of the devices in a set of such devices includes a post 56 having a post end of a different hardness and/or dimension. That is, the alternative hand-held massage device of the present invention may have a post 56 with a post end 58 configured in the manner of the end caps 22/24/26/28 of the device 10 of FIG. 1 so that the patient may switch from one such device of the set to the next in the progression of the treatment of the trigger point. That is, each of the end caps 22/24/26/28 may be permanently affixed to the post 56 of a plurality of the devices 50 and the patient may progress from one device to another rather than from one end cap to another. Color embodiment may be employed with this alternative embodiment with respect to one or more of the device 50, the post end 58 and the end caps.

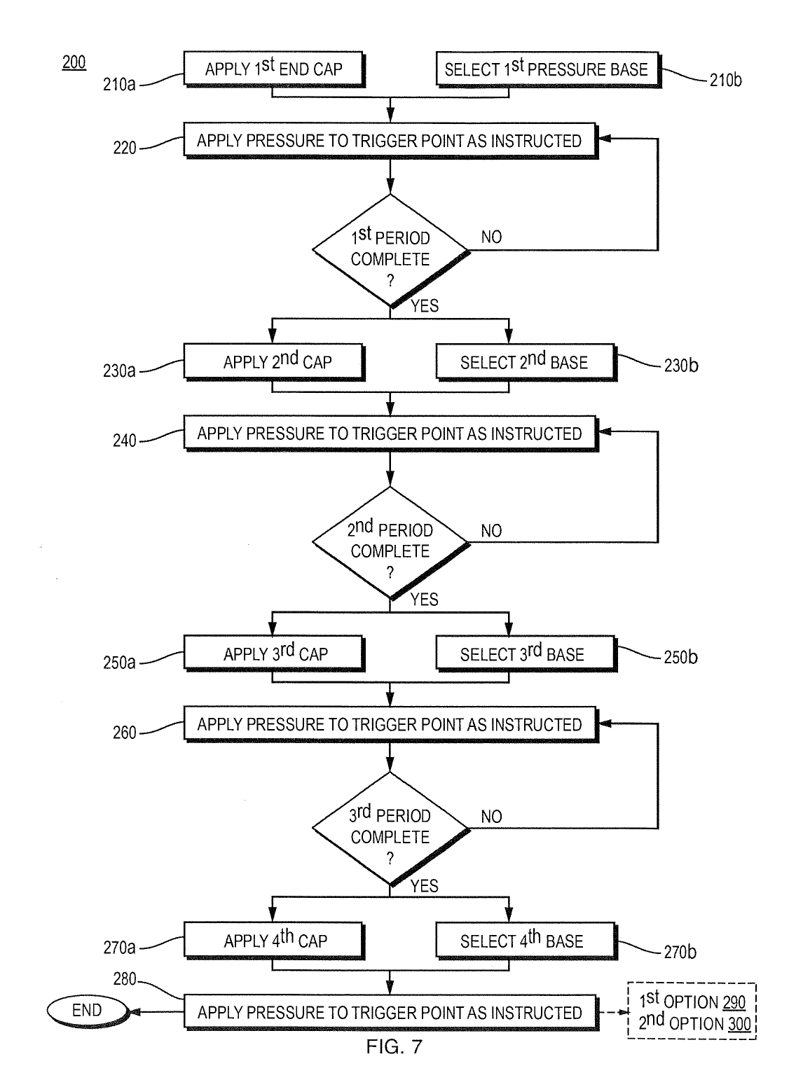

Any of the massage device embodiments described herein can be used in a trigger point treatment method 200 of the present invention represented in FIG. 7. Preferably after consultation with a healthcare giver, a user of the device 10/10′/50/100, selects, or is provided with, one of the devices for the purpose of treating one or more trigger points. For devices 10, 10′ and 50, a first step 210a of the method 200 is to apply the first end cap 22 to the terminal base 20 or 20′ or the post end 58 for the initial trigger point pressure application. It is to be noted that any of the devices may be used for self-treatment or treatment by another. For the device 100, a first step 210b of the method 200 is to select the first pressure base 112 for the initial trigger point pressure application. A second step 220 of the method 200 is to apply pressure to the trigger point using the first end cap 22 or the first pressure base 112 to the location of the trigger point in a manner prescribed or instructed for a selectable length of time over a selectable period of time, which period of time may be one week or more or less than that. The pressure applied should be a function of the particular needs of the person. Suitable medical guidelines established by those of skill in the field of muscle pain treatment may be referenced for that purpose.

For any of devices 10, 10′ and 50, a third step 230a of the method 200 is to remove the first end cap 22 from the terminal base 20 or 20′ or the post end 58 and apply the second end cap 24 to the terminal base 20 or 20′ or the post end 58 for the second trigger point pressure application wherein greater pressure is applied using the device 10/10′/50 at the trigger point location than was applied in step 220. For the device 100, a third step 230b of the method 200 is to select the second pressure base 114 for the second trigger point pressure application wherein greater pressure is applied using the device 100 at the trigger point location than was applied in step 220. A fourth step 240 of the method 200 is to apply pressure to the trigger point using the second end cap 24 or the second pressure base 114 to the location of the trigger point in a manner prescribed or instructed for a selectable length of time over a selectable period of time, which period of time may be one week or more or less than that.

For any of the devices 10, 10′ and 50, a fifth step 250a of the method 200 is to remove the second end cap 24 from the terminal base 20 or 20′ or the post end 58 and apply the third end cap 26 to the terminal base 20 or 20′ or the post end 58 for the third trigger point pressure application wherein greater pressure is applied using the device 10/10′/50 at the trigger point location than was applied in step 240. For the device 100, a fifth step 250b of the method 200 is to select the third pressure base 116 for the third trigger point pressure application wherein greater pressure is applied using the device 100 at the trigger point location than was applied in step 240. A sixth step 260 of the method 200 is to apply pressure to the trigger point using the third end cap 26 or the third pressure base 116 to the location of the trigger point in a manner prescribed or instructed for a selectable length of time over a selectable period of time, which period of time may be one week or more or less than that.

For any of the devices 10, 10′ and 50, a seventh step 270a of the method 200 is to remove the third end cap 26 from the terminal base 20 or 20′ or the post end 58 and apply the fourth end cap 28 to the terminal base 20 or 20′ or the post end 58 for the fourth trigger point pressure application wherein greater pressure is applied using the device 10/10′/50 at the trigger point location than was applied in step 260. For the device 100, a seventh step 270b of the method 200 is to select the fourth pressure base 118 for the fourth trigger point pressure application wherein greater pressure is applied using the device 100 at the trigger point location than was applied in step 260. An eighth step 280 of the method 200 is to apply pressure to the trigger point using the fourth end cap 28 or the fourth pressure base 118 to the location of the trigger point in a manner prescribed or instructed for a selectable length of time over a selectable period of time, which period of time may be one week or more or less than that.

A first optional final step 290 of the method 200 is to apply pressure to the trigger point location using the terminal base 20//20′110 or the post end 58. A second final optional step 300 is to maintain the treatment for an extended time period using at least the terminal base 20/20′/110 or the post end 50, or any one or more of the end caps or pressure bases as deemed suitable.

Alternatively or additionally, the method of treating a person with a trigger point condition can also include the step of testing the sensitivity of the person, such as with a dolorimeter, to determine which end cap or terminal to begin with, change to, continue with and/or end with in the course of the treatment. That is, it may not be necessary to use all end caps/terminals, or even use them in the specific order noted herein. For example, the provider may begin with a relatively softer end cap, skip to the hardest and then revert back to one in between as a function of the person's sensitivity. Also, as noted, the duration of treatment may be varied, including being regular or sporadic, and may include day-long, week-long or even single-treatment sessions. It is to be noted that the invention is not limited thereto.

A kit including a plurality of the massage devices of the present was used to treat a group of seven patients suffering from myofascial pain. Each of the seven patients had been using the Thera Cane™ product prior to using the present invention. The kit included the Thera Cane™ product as the primary body 12 with the terminal base 20. The kit also included the four end caps, the first end cap 22 (softest), the second end cap 24, the third end cap 26 and the fourth end cap 28 (hardest). Each of the patients was instructed to conduct self therapy in accordance with the following protocol.

Protocol:

The patients were instructed to use the primary body 12 with the first end cap 22 on the terminal base 20 during the first week of treatment, with the second end cap 24 during the second week, with the third end cap 26 during the third week, the fourth end cap 28 during the fourth week and the terminal base 20 without any end cap during the fifth week.

Each patient was directed first to apply steady pressure, to a level of some discomfort but not severe pain, for 10 seconds to one minute, until pain lessens then release 10 seconds and take a few deep breaths. Next, steady pressure was applied again, but deeper and with more force to a level of some discomfort but not severe pain, for 10 seconds to one minute, until pain lessens then release 10 seconds and taking a few deep breaths after. Steady pressure was applied for a third time, but still deeper and with more force to a level of some discomfort but not severe pain, for 10 seconds to one minute, until pain lessens then release 10 seconds and taking a few deep breaths again. If the starting end cap used in a particular week did not cause some level of discomfort, the patient could switch to the next harder end cap as the starting one. If an end cap used caused too much pain, the patient could go back to a softer end cap for a longer period before switching to a firmer/harder end cap.

After completion of the five-week period of therapy conducted in accordance with the Protocol, the patients were subjected to physical examination associated with their trigger point pain conditions. The examination revealed that after use of the kit, several of the patients exhibited a reduction in muscle tenderness and less tightness in the muscles. In addition, the seven patients were asked a series of questions regarding the level of myofascial pain they experienced using the kit in comparison to using the Thera Cane™ product alone for a substantially equivalent period of time. The questions and answers to the questions asked are listed in Table 1.

TABLE 1

| | Total number of |
|---|---|
| | patients answering |
| Question | in the affirmative |
| Did you complete the entire five-week protocol? | 7 |
| Did use of the kit with multiple end caps help | 7 |
| your muscle condition? | |
| Did the kit with multiple end caps decrease | 7 |
| your pain or discomfort? | |
| Did the kit with multiple end caps improve | 6 |
| your range of motion? | |
| Were the results of your treatment using the kit | 7 |
| with multiple end caps better than using the There | |
| Cane ™ product alone? | |

The patients provided additional feedback after completing the evaluation. Five of the seven patients felt it was best to start with the first end cap 22, one felt it was best to start with the second end cap 24 and the seventh patient felt it was best to start with the fourth end cap 28. Five of the patients were able to progress to using the terminal base 20 without any end caps, while two patients ended with the fourth end cap 28. Two patients felt that the fourth end cap 28 worked best, two patients felt that the first end cap 22 worked best, and two patients felt that the third end cap 26 worked best to treat their conditions. Additional comments included:
1. “Angling the cap to align better so center is used instead of side.”
2. “I found it was too much with the Thera Cane alone—plus it was harder to pinpoint the spot. The larger caps were more about diffusing the pain, I liked the progression toward the smaller caps.”
3. “Keep the first, second and third end caps longer, covered larger area and was more comfortable to use.”
4. “First end cap was too large, difficult to find trigger point and apply meaningful pressure.”
5. “The end cap definitely helped me use the Thera Cane. I felt too much discomfort using the Thera Cane initially without the end cap, but I am able to tolerate it now.”

The results of the study conducted on patients suffering from myofascial pain trigger points make it clear that the present invention substantially improves the functionality of prior treatment tools by providing gradual, uniform and systematic progression of pressure and release. The device and method of the invention enable the application of pressure in a manner that allows a patient to tolerate the pressure without tensing the relevant muscles and/or causing the locus of pain including a trigger point to become hyperirritable and painful. The device and method also enable the gradual selectable increase in that applied pressure while still preventing the tense response from the patient so that the optimal or desired amount of focused pressure is tolerated. The device enables progressive deep tissue massage in systematic and progressive steps that enhances the treatment of myofascial trigger points in skeletal muscles, as well as other forms of muscle pain.

The device and related method of the present invention enables a user to treat in an effective way the trigger points that exist for people suffering from myofascial pain syndrome. The device is configured to permit the user to apply an appropriate amount of trigger point pressure without also causing muscle tensing. That applied pressure may be increased gradually using pressure applicators of different hardnesses and/or dimensions until the maximum reasonable compression is created while minimizing the possibility of muscle tensing occurring.

The present invention has been described with respect to various examples. Nevertheless, it is to be understood that various modifications may be made without departing from the spirit and scope of the invention. All equivalents are deemed to fall within the scope of this description of the invention.

What is claimed is:

1. A device for treating muscle pain that may include one or more trigger points, the device comprising:
a. a base device including a terminal and means for causing movement of the base device; and
b. one or more end caps removably placeable on the terminal, wherein the one or more end caps are of one or more different physical characteristics than the terminal and each other.

2. The device of claim 1 wherein the one or more different physical characteristics are selected from hardness, dimension and shape.

3. The device of claim 1 wherein there is a plurality of end caps and each of the plurality of end caps is of a different hardness and/or dimension than that of the others of the plurality of end caps.

4. The device of claim 1 wherein the base device is a hand held device having a post and the terminal is a post end.

5. The device of claim 1 wherein the base device is a curved structure.

6. The device of claim 1 wherein one or more of the base device, the terminal of the base device and the one or more end caps are color coded.

7. A kit for treating muscle pain that may include one or more trigger points, the kit comprising:
a. a first device including a terminal and means for causing movement of a first base device; and
b. a second device including a terminal and means for causing movement of a second base device,
wherein the terminal of the first base device has one or more physical characteristics different from one or more physical characteristics of the second base device.

8. The kit of claim 7 wherein the one or more different physical characteristics are selected from hardness, dimension and shape.

9. The kit of claim 7 further comprising a third device including a terminal and means for causing movement of a third base device, wherein the terminal of the third base device has one or more physical characteristics different from the physical characteristics of the first base device and the second base device.

10. The kit of claim 9 wherein the one or more different physical characteristics are selected from hardness, dimension and shape.

11. The kit of claim 7 wherein the first base device and the second base device are hand held devices, each having a post, wherein the terminals are post ends.

12. The kit of claim 7 wherein each of the first base device and the second base device is a curved structure.

13. The kit of claim 7 wherein one or more of the first device, the second device and one or more of the terminals are color coded.

14. A method for treating muscle pain that may include one or more trigger points, the method comprising the steps of:
a. applying pressure to an area where the muscle pain exists using a device having a terminal of a first one or more physical characteristics;
b. releasing the pressure applied with the device having the terminal of the first one or more physical characteristics;
c. changing the terminal of the device so that it is of a second one or more physical characteristics, wherein the second one or more physical characteristics are different from the first one or more physical characteristics; and
d. applying pressure to the area where the muscle pain exists using the device with the second hardness and/or second dimension.

15. The method of claim 14 wherein the first one or more different physical characteristics and the second one or more different physical characteristics are selected from hardness, dimension and shape.

16. The method of claim 14 wherein one or more of the device and one or more of the terminals are color coded.

17. The method of claim 14 further comprising the step of determining sensitivity associated with the muscle pain prior to selecting the terminal to use in applying pressure.

18. The method of claim 14 wherein a time frame for changing the terminal of the device is selectable.

19. A method for treating muscle pain that may include one or more trigger points, the method comprising the steps of:
a. applying trigger point pressure release to a trigger point using a first device having a terminal of a first one or more different physical characteristics; and
b. applying trigger point pressure release to the trigger point using a second device having a terminal with a second one or more different physical characteristics,
wherein the first one or more different physical characteristics the second one or more different physical characteristics are not equal.

20. The method of claim 19 wherein the first one or more different physical characteristics and the second one or more different physical characteristics are selected from hardness, dimension and shape.

21. The method of claim 19 wherein one or more of the first device, the second device and one or more of the terminals are color coded.

22. The method of claim 19 further comprising the step of determining sensitivity associated with the muscle pain prior to selecting the terminal to use in applying pressure.

23. The method of claim 19 wherein a time frame for applying trigger point pressure using either or both of the first device and the second device is selectable.
